(19) United States
(12) Patent Application Publication
(10) Pub. No.: US 2025/0054593 A1
(43) Pub. Date: Feb. 13, 2025

(54) ORCHESTRATED PERSONALIZED THERAPY SYSTEMS AND METHODS

(71) Applicants: Ryan BANDOUVERES, Horsham, PA (US); Michael HARRISON, Horsham, PA (US); Midhun KRISHNA, Horsham, PA (US); Chirag SHAH, Horsham, PA (US); Cody SIMS, Horsham, PA (US); Haneya YUNUS, Horsham, PA (US); Janssen Biotech, Inc., Horsham, PA (US)

(72) Inventors: Ryan Bandouveres, Horsham, PA (US); Michael Harrison, Horsham, PA (US); Krishna Midhun, Horsham, PA (US); Chirag Shah, Horsham, PA (US); Cody Sims, Horsham, PA (US); Haneya Yunus, Horsham, PA (US)

(21) Appl. No.: 18/720,427

(22) PCT Filed: Dec. 16, 2022
(86) PCT No.: PCT/US2022/081771

§ 371 Date: Jun. 14, 2024

Related U.S. Application Data
(60) Provisional application No. 63/291,062, filed on Dec. 17, 2021.

(51) Int. Cl. G16H 20/00; G16H 10/60

(52) U.S. Cl. CPC G16H 20/00; G16H 10/60

(57) ABSTRACT

Disclosed are devices, systems, methods, and computer program products for orchestrating personalized therapy order management, including managing treatment workflows.

### CROSS-REFERENCE TO RELATED APPLICATION(S)

This application claims the benefit of U.S. Provisional Patent Application No. 63/291,062, filed Dec. 17, 2021, and incorporated by reference herein in its entirety.

### TECHNICAL FIELD

The present disclosure is directed to systems, methods, devices, and computer program products for orchestrating personalized medicine therapies orders.

### BACKGROUND

Personalized medicine is a medical treatment model that provides medical decisions, practices, therapies, interventions and/or products specifically tailored to an individual patient based on the individual's predicted response or risk of disease. Immunotherapy is a form of personalized medicine rapidly emerging for disease treatment and prevention. Immunotherapy includes, for example, engineering a modified version of a patient's own biological material (e.g., immune cells) and reintroducing the engineered biological material into the patient to initiate and/or supplement the patient's immune response to combat a disease. For example, CAR T-cell therapy is a cancer treatment that involves the extraction of T-cells from a patient's blood and modifying the cell surface to include chimeric antigen receptors that facilitate the production of a chemical to attack tumor cells. These and other individualized approaches allow for the provisioning of customized therapies based on calculated predictions for how treatments may work best for a particular patient.

Although these treatments are revolutionizing disease treatment, a complex chain of processes must be undertaken in order to process, generate, and deliver the therapeutics ultimately administered to the patient. Personalized medicine supply chains are fraught with logistical challenges, scheduling challenges, stakeholder coordination challenges, monitoring and tracking challenges, and other related problems that impede the ability to implement personalized medicine on a large or global scale.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1 shows a diagram of an environment for supply chain management of a personalized therapy, including an enterprise software platform for orchestrated personalized therapy order management configured in accordance with the present technology to facilitate data transactions between computing devices of external systems.

FIGS. 2A and 2B show diagrams illustrating example processes in a supply chain for implementing a personalized therapy.

FIG. 3 shows a diagram depicting an example embodiment of the set of application program interfaces APIs for the enterprise software platform of FIG. 1.

FIGS. 4A and 4B show diagrams of an example user interfaces associated with an API to manage patient data transactions configured in accordance with the present technology.

FIG. 5 shows a diagram of an example implementation for execution an API to manage ordering data transactions configured in accordance with the present technology.

FIG. 6 shows a diagram of illustrating example data transactions managed by an example embodiment of APIs of the enterprise software platform of FIG. 1.

FIG. 7 shows a diagram of a method for creating a treatment workflow by the enterprise software platform of FIG. 1 based on an order request submitted by an external system.

FIG. 8 shows a block diagram of an example embodiment of a computer device.

### DETAILED DESCRIPTION

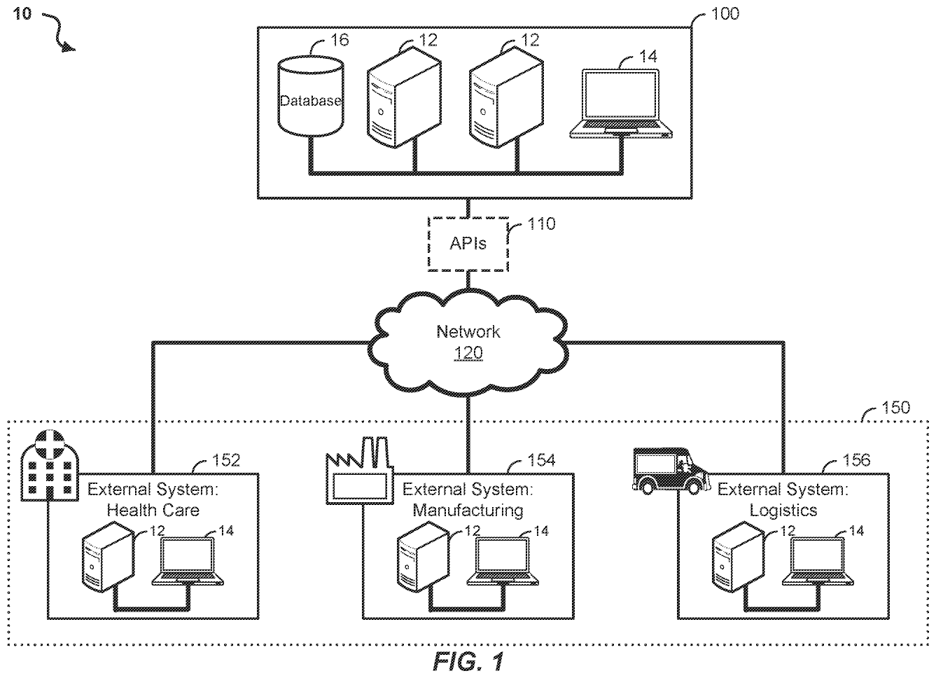

The present disclosure is directed to systems, methods, devices, and computer program products for orchestrated personalized therapy order management, particularly in managing treatment workflows. For example, in the implementations described below, the disclosed technology for orchestrated personalized therapy order management includes specialized application program interfaces for an enterprise software platform, which is primarily described in the context of personalized medicine treatment (CAR T-cell therapy). It will be appreciated, however, that the disclosed technology can be utilized with a variety of other cell and gene therapies, personalized medicine treatments, as well as a variety of other medical or non-medical systems. Furthermore, a person skilled in the relevant art will understand (i) that the technology may have additional implementations than illustrated in FIGS. 1-8 and (ii) that the technology may be practiced without several of the details of the implementations described below with reference to FIGS. 1-8.

### Overview

Advanced therapies, particularly personalized therapies, have forced a paradigm shift in medical supply chain management. Instead of coordinating several components for large batches of “one-size-fits all” therapies, such as vaccines, blockbuster drugs, etc., pharmaceutical manufacturers and health care professionals must now manage a highly complex supply chain of personalized components for production of an individualized therapy in very small batches-sometimes as small as one therapy per patient—and at growing volumes.

Presently, supply chains for personalized therapies such as CAR T-cell therapy are distributed in a manner built around patients and donors, but must also account for activities and constraints associated with the other supply chain stakeholders, particularly (i) manufacturers of biological material, (ii) transporters of materials, and (iii) health care providers (HCPs) and their settings, including hospitals, laboratories, and clinics. As such, supply chains are highly complex structures to view, track, and manage (in real-time) for each supply chain stakeholder. For example, obstacles exist for accurate and efficient accounting of the chain-of-identity (COI) and chain-of-custody (COC) of materials to ensure the safety of the end personalized therapy product across the entire product cycle from “vein to vein,” i.e., from the earliest event such as extraction of biological material from a patient for creating the personalized therapy to a last event such as delivery of the personalized therapy for treating the patient's disease or condition.

In the “vein to vein” lifecycle to develop and deploy a personalized therapy, there are dozens, if not hundreds, of stakeholder handoffs that must be enabled, tracked, and documented for every patient product. Each product journey, i.e., the transition of a material or materials from one stakeholder or process to another, is likely to be highly variable and susceptible to schedule changes and unexpected events that can disrupt supply chain flow, and thereby devastate the personalized therapy for a patient. For example, it is presently very difficult to align the starting material collections process with manufacturing capacity such that precious cellar material is not lost and manufacturing capacity is utilized and not overbooked or over extended. Also, when a disruption in the supply chain occurs, the supply chain must be able to adapt to unexpected events; however, the ability to reschedule events is not feasible without integrated tools with connected data flows and orchestration, particularly orchestration of the entire supply chain-including logistics—from order to treatment, with a single source of real-time transparency and visibility.

One approach aimed to address these and other challenges to meet the demands for personalized medicine is a cloud-based, individualized medicine software platform technology, developed by Vineti, Inc., the assignee of the present technology. Vineti's platform technology is an aPaaS (Application Platform as a Service), which is a purpose-built enterprise software service providing a common technological foundation to meet each individual client needs through end-user administrator configuration, and not by creating standalone custom builds. Aspects of the platform technology are discussed in U.S. Patent App. Publication No. US 2020/0177386 and U.S. Patent App. Publication No. US 2021/0280287, both of which are incorporated herein by reference in their entireties. For example, the exemplary cloud-based platform technology executes a range of operations related to data communications and transactions to coordinate events that occur in an individualized medicine supply chain, e.g., including but not limited to ordering and scheduling of biological material extraction, material and/or kit delivery, material and/or kit tracking, automated courier scheduling, intermediary and final product preparation, capacity and resource management, etc. In some aspects, the cloud-based platform technology is configured to facilitate interactions between an individualized medicine system and one or more nodes of a blockchain configured to store event data and identification data corresponding to transactions that occur along an individualized medicine supply chain.

Traditionally, pharmaceutical manufacturers or HCPs managed their roles in the practice of generalized medicine using custom software. Yet, as personalized medicine continues to grow, custom software architecture has become incapable to facilitate personalized medicine treatment practices and address, let alone satisfy, supply chain stakeholder needs. As such, enterprise software has become a more practical model to build a personalized medicine supply chain. The above-referenced platform technology is one example of an enterprise software platform developed for personalized medicine management.

Generally, enterprise software is a collection of computer programs that have common applications (e.g., business- or commerce-focused), modeling tools for managing how an organization works, and development tools for building applications unique to the organization. Enterprise software is architected for addressing large-scale problems across an ‘enterprise’ (multiple stakeholders in a community, for example), rather than individualized or single-entity problem, for the sake of improving the enterprise's productivity and efficiency through software- and network-based support functionality. As such, distinctive features for individual end-users of enterprise software are difficult to create and manage. In contrast, custom software is software that is specially developed for a particular or individualized organization or user, i.e., it is designed to meet the customer's particular, individualized preferences and expectations.

For enterprise software and custom software systems, data interconnectivity between different software programs or computers can be addressed through the use of application programming interfaces. An application programming interface (API) is a type of software interface that provides a connection between computer programs (software applications) on the same or different computers. In general, an API is often made up of different parts, such as subroutines, methods, requests, or endpoints, which act as tools or services that can be called for use. The specification of an API specification defines these calls, but an API is typically designed to hide the internal details of how a system works, while exposing parts of the system useful or needed for making later changes. For example, an API for file input/output might publish a function that copies a file from one location to another without disclosing any information about the file system operations behind the scenes.

Yet, data communications between computers in an enterprise software platform is becoming more complex. For example, with increasing numbers of databases, cloud applications, and endpoints, and clients located globally that demand information in real time, information within an enterprise software platform is constantly being exchanged. These complexities are even more severe when attempting to integrate an enterprise software platform with another enterprise software or custom software platform, which can be even further compounded for a supply chain management software platform, e.g., particularly one dedicated to personalized therapy management where the health and life of stakeholders may be dependent about the timing and seamless orchestration of each point and transition in the supply chain.

For example, analysts in the medical treatment industry have widely noted that complexity of the supply chain is the critical limiting factor for advancing personalized therapies, particularly for cancer treatment. Complexity has led to an extremely high cost of goods sold and hesitation to utilize certain cancer therapies, like CAR T-cell therapies, beyond end-of-line patients and patient drop-offs along a treatment journey. Thus, while the underlying medical science behind cell and gene therapies continues to make rapid, remarkable progress, the utilization and implementation of new therapies to treat patients is falling farther and farther behind, when viewed on a large and broad scale. Moreover, the global pandemic caused by the proliferation of COVID-19 has further exacerbated the complexities in personalized therapy management, for example, with one-third of cell therapy companies reporting manufacturing delays or a complete halt to operations in 2020 and 50 percent of cell gene therapy companies reporting a reduction in engagement with HCPs, clinical sites, and patients. Thus, scaling out the production and delivery of high-value, deeply personalized products, such as autologous and allogeneic cell and gene therapies, as well as personalized cancer vaccines, requires significantly more simplification and standardization if the promise these therapies hold will be realized for the benefit of patients everywhere.

The present technology provides systems, methods, devices, and computer program products for orchestrated personalized therapy order management, particularly in managing treatment workflows. In example embodiments, the disclosed technology provides personalized therapy treatment workflow management, including patient management, order management, and scheduling management, with track and trace solutions that facilitate patient initiation while (i) synchronizing order event data with integrated systems, (ii) minimizing protected health information (PHI) footprint across an information technology (IT) ecosystem, and (iii) simplifying a multi-portal healthcare provider experience.

Further advantages of the disclosed technology include direct importation of patient data from an external system, which is expected to eliminate high-risk errors in mis-capture of critical patient data and simplifying the order creation process. Using the disclosed technology, order management can be entirely managed outside of track and trace orchestration system while synchronizing order data, patient data, and schedule data entirely via the system's sophisticated APIs. Moreover, personalized therapy order management may be embedded directly in external systems, such as an electronic health record (EHR) portal and a customer relationship management (CRM) portal, to provide full life cycle order management including creation, scheduling, submitting, approving, and rescheduling in a seamless single-portal experience.

### Personalized Therapy Treatment Workflow Management System

FIG. 1 shows a diagram of an environment 10 for supply chain management of a personalized therapy, in which an enterprise software platform 100 for orchestrated personalized therapy order management configured in accordance with the present technology can facilitate data transactions between computing devices of one or more external system(s) 150 for the development and treatment of a patient with a personalized therapy treatment product and process. Herein, the enterprise software platform for orchestrated personalized therapy order management may be referred to as the “enterprise software platform 100” or simply the “platform 100.”

The environment 10 includes one or more server computer devices 12 and one or more server and/or client computer devices 14, which are associated with the enterprise software platform 100 and associated with the external system(s) 150. The one or more server computer devices 12 and/or the one or more client computer devices 14 can generate, curate, track, monitor, and store data associated with transactional processes of the personalized therapy supply chain, including events associated with ordering, manufacturing, delivering, and treatment of cell therapies and gene therapies for patients. In some embodiments, the enterprise software platform 100 includes one or more databases 16 configured to store, organize, and manage large collections of the data. Similarly, the external system(s) 150 may include one or more databases 16 for some embodiments (not shown). In implementations, for example, the computer devices 12, 14 and database(s) 16 of the enterprise software platform 100 and the computer devices 12, 14 (and database(s) 16) of the external system(s) 150 can communicate in the environment 10 via a network 120, e.g., such as the Internet.

The computer devices 12, 14 of the enterprise software platform 100 can perform a range of operations related to data corresponding to the supply chain events, including but not limited to point-of-care collection (e.g., collection of samples that meet threshold quality standard requirements), tracking supply chain events (e.g., as relates to manufacturing a personalized therapeutic for a patient), optimizing resource utilization (e.g., fulfilling manufacturing orders), security and compliance (e.g., ensuring data quality, data validation, and data encryption mechanisms are implemented), ordering and scheduling events (e.g., scheduling of sample collections, manufacturing, administration of medicine, etc., and scheduling couriers, raw material delivery, tracking kits and materials and raw materials, etc.), reporting tasks (e.g., reporting quality findings, compliance activities, chain of identity information, chain of custody information, etc.), and/or facilitating system integrations (e.g., data integrations, data modifications, insight procurement, etc.).

As shown by the example of FIG. 1, the enterprise software platform 100 can be in data communication with different external systems associated with supply chain orchestration, including but not limited to a health care facility system 152, a manufacturing facility system 154, and/or a logistics facility system 156. Each of these example external systems 152, 154, and 156 can include one or more server computer devices 12 and one or more client computer devices 14; and the external system(s) 150 can include a single or multiple health care facility system(s) 152, a single or multiple manufacturing facility system(s) 154, and/or a single or multiple logistics facility system(s) 156 in data communication with the enterprise software platform 100 within the environment 10.

The enterprise software platform 100 can include a plurality of software modules to orchestrate a personalized therapy treatment among multiple stakeholders in personalized therapy treatment supply chain. In some examples, the plurality of software modules, operable on the computer devices 12, 16, and/or 14 of the enterprise software platform 100, include a supply chain management module, a chain of identification and chain of custody module, a systems integration module, and an analytical module. Some example embodiments and implementations of these modules of the enterprise software platform 100 are described in U.S. Patent App. Publication No. US 2020/0177386 (which was previously incorporated herein by reference), e.g., with respect to the supply chain optimization module 110, the custody & identification module 130, the systems integration module 140, and the analytics module 150. The enterprise software platform 100 can include variations of these modules and/or other modules. Some other example embodiments and implementations of the modules of the enterprise software platform 100 are described in U.S. Patent App. Publication No. US 2021/0280287 (which was previously incorporated herein by reference).

As will be discussed further below, the enterprise software platform 100 provides a set of APIs 110 to facilitate data transactions for personalized medicine therapies orders, which can alleviate the various challenges along the various points along the personalized medicine supply chain, such as logistical challenges, scheduling challenges, stakeholder coordination challenges, monitoring and tracking challenges, and other related problems. One key advantage of implementation of the disclosed technology is that the set of APIs 110 can create and maintain a unique, robust Chain of Identity (COI) for an ordered personalized therapy, despite the multiple stakeholders and diverse external systems 150 that may be within the environment 10.

In personalized therapeutics, safety and efficacy rely on the right patient receiving the right treatment. The process of tracking and matching materials in the supply chain is driven by COI, which is the permanent, transparent association of a patient and/or donor's unique identifiers to their tissue and/or cells from order through collection, manufacturing, administration, and post-treatment monitoring. Pairing COI with COC, which is the permanent data capture of tracking and handling information for a therapy at every step, can provide a truly traceable, trustworthy process.

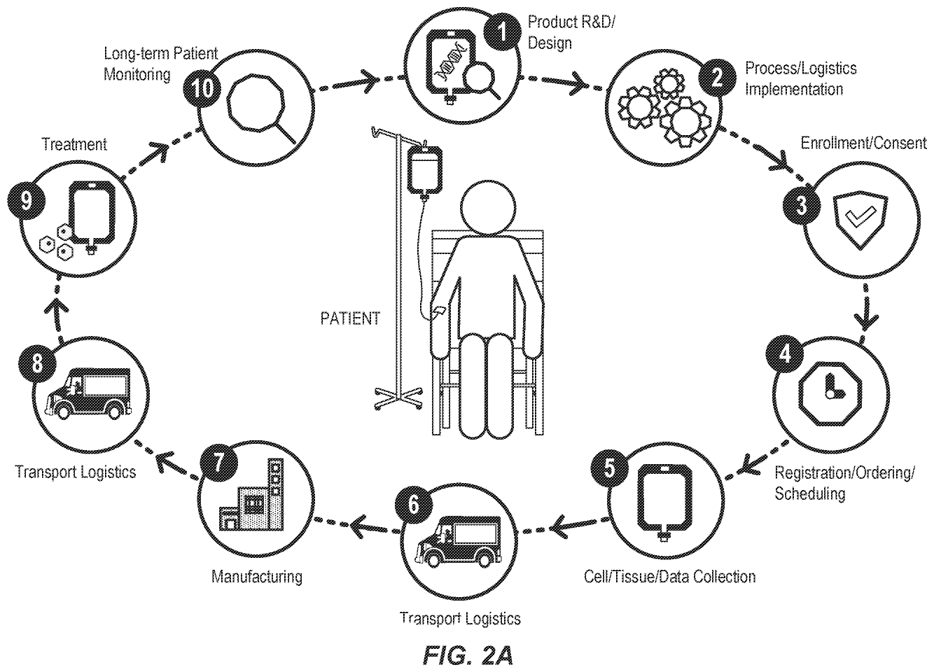

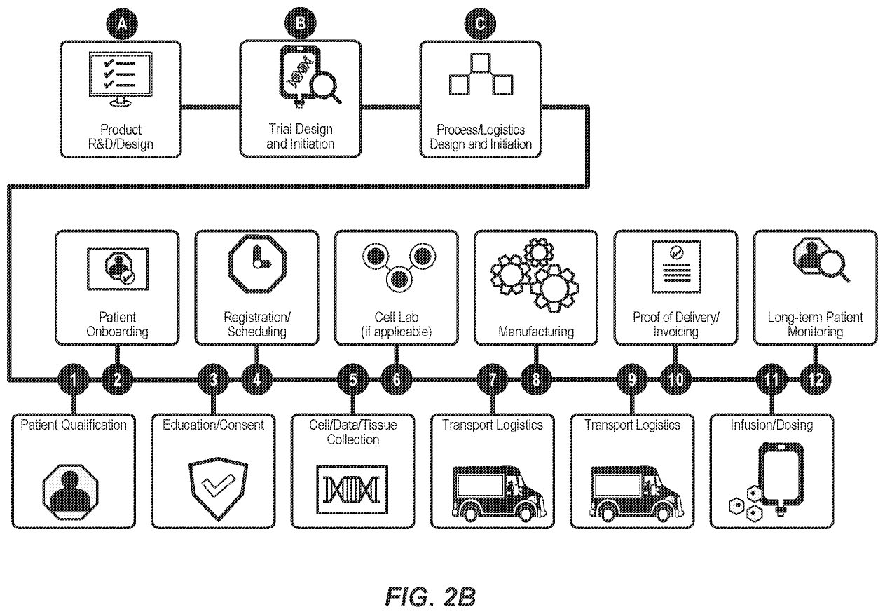

FIG. 2A shows a diagram illustrating an example process for implementing a personalized therapy, e.g., depicting various stakeholders and/or transactions in a supply chain across a “vein to vein” to therapy cycle. For example, when a personalize therapeutic product and procedure is devised for a patient (event 1), various concurrent and sequential actions must begin to implement the personalized therapy process (event 2) to coordinate the logistics of the personalized therapy. For example, the patient is enrolled and consents to the prescribed personalized therapy by the HCP (event 3), after which registration, ordering, and scheduling commences (event 4). The scheduling, for example, can set dates for key events, such as the extraction of biological material from the patient (event 5), the transportation logistics (event 6) for providing the biological material to the personalized therapy product manufacturer for creation of the product (event 7), the transportation logistics (event 8) providing for the personalized therapy product to the treatment facility (event 9), and the long-term patient monitoring (event 10) to ensure the effectiveness in the personalized therapy treatment the patient's disease or condition.

FIG. 2B shows a diagram illustrating another example process for implementing a personalized therapy. The diagram of FIG. 2B illustrates events prior to implementation of a personalized therapy, including product research, design and development (item A), product trial design and initiation (item B), and process and logistics design and initiation (item C). The diagram of FIG. 2B also illustrates a modified sequence of events in the personalized medicine treatment process from that of FIG. 2A.

At a basic level, Chain of Identity is enabled by a COI Identifier (COI ID), a set of numbers and/or characters that is the overarching “ID badge” for each patient and key materials related to their product. The COI ID must link a therapy to the intended patient and protect patient privacy. The COI ID is used in multiple places on the patient journey, from medical records to in-process drug labels.

Table 1 (shown below) illustrates an example embodiment of the single COI ID comprising a core identifier (core ID), a treatment identifier (treatment ID), and a specimen identifier (specimen ID). In this example embodiment, the core ID includes a nine-character code beginning with a three-letter company identifier that is unique to a company/sponsor, followed by a six-digit core patient ID, which is unique to a patient for all of their product journeys. In this example, the treatment ID includes a five-character code, which trails the core ID (that may optionally include a dash “-” character), beginning with a three-letter product identifier that is unique to a product or therapy, followed by a two-digit order identifier, which indicates a specific treatment order cycle. Finally, in this example, the specimen ID includes a six-character code, which trails the specimen ID (that may optionally include a dash “-” character), beginning with a two-letter procedure identifier that indicates a procedure type and/or grouping of specimens, followed by a two-digit run identifier that indicates a specific session/run of a procedure type, and followed by a two-digit item identifier that indicates and item number produced in a session/run.

TABLE 1
CMP123456-PRD01-FP0201
Core COI ID
Treatment ID
Specimen ID
Core COI ID
Treatment ID
Specimen ID

| Company | Core Patient | Product | Order | Procedure | Run | Item |
|---|---|---|---|---|---|---|
| identifier | ID | identifier | identifier | identifier | identifier | identifier |
| Unique to a | Unique to a | Unique to a | Indicates a | Indicates a | Indicates a | Indicates |
| company/ | patient for all | product | specific | procedure | specific | item number |
| sponsor | their product | (or therapy) | treatment | type and/or | session/run of | produced in a |
| | journeys | | order cycle | grouping of | a procedure | session/run |
| | | | | specimens | type | |

Table 2 (shown below) illustrates another example embodiment of a COI ID structure, comprising the product identifier of the treatment ID, the core patient ID of the core ID, a process identifier (unique to a specific treatment associated with an order cycle), and a check digit. In this example embodiment, the COI ID includes the product identifier, the core patient ID, the process identifier, and the check digit in order and separated by a period, dot, or other character to indicate separation. The product identifier includes a three-character code of base 36 characters, i.e., 0-9 and A-Z; the core patient ID includes a six-character code of base 36 characters; the process identifier includes a three-character code of hexadecimal characters; and the check digit includes a single character of decimal characters. Table 2 illustrates the structure for this example of the COI ID for the COI ID being: PRD.PAT.123.E45.9.

TABLE 2
Example: PRD.PAT123.E45.9

| PRD | | E45 | |
|---|---|---|---|
| Product | PAT123 | Process | 9 |
| identifier | Core patient ID | identifier | Check digit |
| Unique to a | Unique to a | Unique to a | Checksum |
| product | specific patient | specific treatment | calculated from |
| (or therapy) | for all their | associated with an | preceding part of ID |
| | product journeys | order cycle | to verify validity |

### APIs of the Personalized Therapy Treatment Workflow Management System

For a personalized therapy, there are a variety of stakeholders who want to create patient orders and patient schedules for personalized therapies in their own external systems, and then send the patient data, order data, and/or scheduling data over to the enterprise software platform 100 for the enterprise software platform 100 to then manage through the whole supply chain. While it could be ideal to create a singularly-adopted software system that every stakeholder entity utilizes to unify the format to which data is entered, formatted, processed, communicated, and stored, such a singularly-adopted system is not realistic in the present health care provider, pharmaceutical and biologics development, and materials logistics environments. Instead, most stakeholders have developed their own internal software systems to serve their own needs and do not wish to risk disruptions by changing, let alone replacing, their systems that they rely on. Presently, most personalized therapy supply chain stakeholders wish to continue utilizing their own custom systems and data formats within the supply chain. Therefore, for the enterprise software platform 100 for orchestrated personalized therapy order management to successfully manage the orchestration of the supply chain and facilitate data transactions between stakeholders seamlessly, the enterprise software platform 100 must provide software interfaces specially tailored to the needs of the personalized therapy supply chain.

In various implementations, the enterprise software platform 100 provides an application platform as a service (APaaS) that bridges the gap between all the complex processes involved in personalized therapy manufacturing, including COI, COC, ordering, material collection, manufacturing, courier logistics, as well as capacity management, all the way through patient intake of the personalized therapy. The APIs 110 of the enterprise software platform 100 enable authorized third-party applications and computers to programmatically enter and/or retrieve patient and treatment data, including permissible data about the patient (patient data), permissible data about the personalized therapy product and/or process order (order data), and permissible data about the schedule of events in the development and treatment of the patient with the personalized therapy (scheduling data). The APIs 110 of the enterprise software platform 100 are configured to be universally applicable to the various custom applications that may be built on different platform frameworks, such that the APIs 110 provide a standard set of data able to work with the data objects and relationships of the enterprise software platform 100.

One aspect of the APIs 110 is authentication. The enterprise software platform 100 requires authentication of external systems (and users) for any system interconnectivity and data communications. For example, the APIs 110 of the enterprise software platform 100 can require an access token associated solely with the platform 100 for making authorized requests. In order to integrate with the enterprise software platform 100, the external system must register and configure the external software application using client credentials that are supplied by the enterprise software platform 100 (e.g., upon request and scrutiny). The enterprise software platform 100 will generate and provide client IDs and secrets in a secure manner for each sandbox and live environment. Once set up, the external system's software application can exchange these credentials for the access token that can subsequently be included in request headers to authorize API calls by the external system. Once an access token is granted, the external software application should include the access token in headers of requests to the APIs 110 of the enterprise software platform 100 to obtain authorization.

In an example implementation of authentication, an authentication server of the enterprise software platform 100 will process the access token request by validating the provided client ID and secret and returning the access token in the response upon successful validation. In some implementations, for example, the access tokens can expire after a predetermined time period (e.g., seconds) after the token is obtained. For example, an access token with an expiration field having a value of 300 would expire in 300 seconds from when the response was generated. Upon expiry, the external software application can obtain a new access token using the same process.

In some embodiments of the set of APIs 110 of the enterprise software platform 100, the set of APIs 110 allows the third-party applications and computers to perform queries and mutations using GraphQL. GraphQL is a query language that allows the caller to request an exact set of fields from data objects of the enterprise software platform 100 to be returned in the response body. GraphQL also can provide users of the enterprise software platform 100 the flexibility to query a sub-set of data from an API of the set of APIs 110, e.g., in contrast with just returning a pre-defined large dataset and/or field(s), as is currently the case with the query language REST. This feature of the API 110 can provide greater query flexibility and less over-fetching than conventional REST-based APIs.

Another key aspect of the APIs 110 is data type identification. The set of APIs 110 of the enterprise software platform 100 handles the calling and tracking of all external IDs, such as patient medical record numbers managed by entities of the external systems 150, e.g., such as health care facility system 152. In order to integrate external and third-party systems with the enterprise software platform 100, currently used external identifiers are mapped to internal identifiers used within enterprise software platform 100 with a resource identifier data structure (also referred to as a “Resource Identifier”). Resource Identifiers are wrappers that allow an object to be referenced within enterprise software platform 100 that has been referenced using the same identifier within an internal application. Resource Identifiers can be used by every internal component of the enterprise software platform 100 to call different types of IDs. For example, this allows the enterprise software platform's 100 internal IDs to be added to the system without having to change existing request data. When defining Resource Identifiers, an identifier type parameter defines the type of identifier, such as a medical record number, a patient ID, or a site ID, and the identifier type parameter provides the identifier itself.

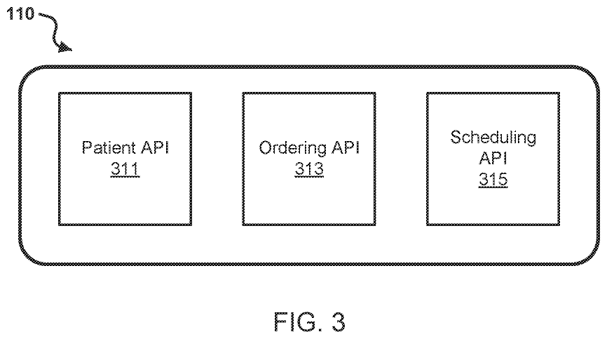

FIG. 3 shows a diagram depicting an example embodiment of the set of APIs 110 for the enterprise software platform 100. The set of APIs 110 include at least three individual APIs that control data transactions between various entities of the external system(s) 150 through the enterprise software platform 100: a Patient API 311, an Ordering API 313, and a Scheduling API 315.

The Patient API 311 interfaces between the enterprise software platform 100 and the external system(s) 150 to enable the creation of patients and update of details for existing patients in the platform 100. In some implementations, for example, the Patient API 311 can be used in an ordering workflow managed by the enterprise software platform 100 to add new patients and update details about patients, e.g., such as date of birth, gender, and weight for existing patients, from the external system(s) 150. In the enterprise software platform 100, a patient data object describes a patient (organizes patient data), including patient ID, patient details, and associated ordering institution. Yet, patients are typically identified by a unique ID provided by a client of the external system(s) 150 (e.g., any or multiple HCP clients); and the Patient API 311 parses the data from the external system(s) 150 to identify and links the unique ID of the external system(s) 150 to the unique identifier (and patient data) within the enterprise software platform 100. In implementations, for example, after a patient has been added to the enterprise software platform 100 with the patient data required by the external system organization, orders can be created for that patient using the external system 150 via the Ordering API 313.

In some implementations of the Patient API 311, for example, the Patient API 311 can be configured to require just the patient ID from the external system 150, and thereby not require patient details or the ordering institution. In implementations where patient details are provided by the external system 150, the Patient API 311 can set parameters associated with patient details that will be linked to the patient ID, including but not limited to the patient's birthdate, name, biological sex and/or identified gender, custom health data (such as weight, height, etc.), and/or contact information (such as email address, phone number, etc.). Also, the Patient API 311 is configured to receive partial information associated with a patient to update an existing patient.

In some implementations of the Patient API 311, for example, the Patient API 311 can be configured to require the patient ID and the associated ordering institution (site) information from the external system 150. In such implementations, for example, this provides a protective layer of security such that users not affiliated with the ordering institution may be restricted from viewing information about the patient, i.e., other entities of the external system 150 may only be able to view information about the order corresponding to the patient ID, without any ability to view information about patient.

The Ordering API 313 interfaces between the enterprise software platform 100 and the external system 150 to enable the creation and canceling of orders in the supply chain. In the enterprise software platform 100, an order data object describes a treatment order for a personalized therapy, which can include patient data, product (therapy) data, and supply chain milestone data (e.g., such as contact information associate with a milestone, site, location, and/or region associated with a milestone, activity associated with a milestone, date/time associated with meeting a milestone, etc.).

The enterprise software platform 100, based at least upon the Ordering API 313, is able to address the complex technical challenges in being able to select the correct, desired workflow by the client when an order request is submitted to the platform 100. For example, an external user (e.g., a user of the health care facility system 152, a user of the manufacturing facility system 154, and a user of the logistics facility system 156) typically has many ordering workflows, which can depend on location, the type of therapy, the quality requirements, the different locations, etc.—a client may have dozens of different ordering workflows that a user, such as an ordering nurse, may need to be able to select from. To illustrate, in some implementations, for example, when an ordering nurse is creating an order for a personalized therapy for a patient, the ordering nurse is presented a list of workflows on a user interface to be selected upfront, where these workflows are typically widely differentiated, e.g., having different events or event parameters. An example of one differentiation is the inclusion of a cryopreservation site within the personalized therapy treatment workflow: does a material for the personalized therapy product go directly to the manufacturing site or does it take a pit stop at a cryopreservation site to be cryo-frozen before going to manufacturing site? With large amounts of workflow, there are large amounts of information that some stakeholders should not be able to access. Yet, protecting the information for certain stakeholders is not possible if an API is merely designed to transfer data between computing systems. The Ordering API 313 addresses these problems and overcomes technology challenges to automate the selection of the most appropriate workflow or workflows to that particular user based on the information that the enterprise software platform 100 receives from the external system 150 via the Ordering API 313.

In some embodiments, for example, the Ordering API 313 is configured to automate workflow selections for actions in the personalized therapy development and treatment supply chain by utilizing criteria, referred to as workflow variants, that are configurable by the user of the external system 150 (e.g., on the external system 150 user interface) to include attributes of the personalized therapy development or treatment process to select the most appropriate workflow (stored by the enterprise software platform 100). In some implementations, for example, the workflow variants can include a finite number of options, where an administrative user of the external system 150 can pre-select certain variables from a list such that the enterprise software platform 100 can construct a maximum amount of workflow permutations from the selected variables. The Ordering API 313 can manage data formatting of the possible workflow options for subsequent presentation (display) on the user interface of the external system 150 so that an end user may select the desired, available attributes of a valid workflow. As an example, for a workflow variant pertaining to cryopreservation of a material in a personalized therapy supply chain, the administrative user could be prompted to select between or among local or centralized cryo-storage and local or centralized label printing for the material. If the administrative user selects local cryo-storage and local or central printing for the material, the enterprise software platform 100 can create two workflows for the Ordering API 313 (e.g., translatable from string data) to implement during the personalized therapy development and treatment supply chain management by that end user: ‘cryopreservation’:‘local’,‘printing’:‘local’ and ‘cryopreservation’:‘local’,‘printing’:‘central’.

In implementations for creating an order, for example, the Ordering API 313 enqueues an order created from the external system 150 to be managed by the enterprise software platform 100 for a personalized therapy for a patient. Once enqueued, the client-side software application will receive success and/or error notifications, which in some examples includes providing notifications asynchronously via HTTP POST to a preconfigured webhook endpoint provided to enterprise software platform 100. For example, a successful response indicates the order was enqueued for creation successfully and contains a transaction ID that can be used to correlate subsequent success/error notifications with the original order creation request to which they pertain.

In some embodiments of the Ordering API 313, for example, the Ordering API 313 can be configured as an asynchronous API that responds to requests with event notifications sent to a webhook. In implementations using GraphQL, for example, the GraphQL response to a request to the Ordering API 313 contains a Transaction UUID that can be used to correlate Ordering API requests with event notification responses. A Transaction UUID is a unique identifier related to a specific API request that is sent to an API of the set of APIs 110, e.g., such as the Ordering API 313, which can be used to distinguish multiple API requests sent at different times to the Ordering API 313. This transaction UUID can be configured to appear in the event notification payload.

In implementations for canceling an order, for example, the Ordering API 313 can enqueue a request to cancel an existing order from the external system 150 to be executed by the enterprise software platform 100. Once enqueued, the client-side software application will receive success and/or error notifications, which in some examples includes providing notifications asynchronously via HTTP POST to the preconfigured webhook endpoint provided to the enterprise software platform 100. For example, a successful response indicates the cancelation request was enqueued successfully and contains a transaction ID that can be used to correlate subsequent success/error notifications with the original cancelation request to which they pertain.

The Scheduling API 315 interfaces between the enterprise software platform 100 and the external system 150 to enable creation, update, and reception of milestone dates from clients of the external system 150 by a schedule request. In the enterprise software platform 100, a schedule object represents the order timeline and is associated with an order that was created via the Ordering API 313. In some embodiments of the Scheduling API 315, for example, the Scheduling API 315 can either use the order object or the treatment object to reference a patient's order or treatment, but not both at the same time.

In some implementations of the Scheduling API 315, the schedule request can include a requested change to the original timeline without approval (e.g., pending request). In example implementations when ordering and manufacturing capacity are managed in a different system outside of the enterprise software platform 100 (i.e., the external system 150), for example, the Scheduling API 315 can accept collection and manufacturing dates, then recalculate the rest of the milestone dates and create the subsequent shipments for the order based on the workflow configuration.

Milestones are points in time where specific events happen. Each treatment workflow is configured with a set of milestones that are used to calculate a patient's treatment timeline from collection to infusion. The enterprise software platform 100 can associate (e.g., assign) milestones with shipment legs and sites. The enterprise software platform 100 can then make calculations based on the durations between each milestone to create an accurate timeline (schedule) for a treatment.

Further details of the Patient API 311, the Ordering API 313, and the Scheduling API 315 are discussed below.

### Patient API

In some clinical and commercial settings for the example enterprise software platform 100, the system that originates patient data is external to the platform 100, e.g., such as the health care facility system 152. Clinical settings and scenarios refer to therapies that are undergoing clinical trials and/or not yet approved by a regulatory agency, such as the U.S. Food and Drug Administration (FDA), e.g., in which the manufacturing process of the therapy may still possess variability as the therapy developer learns more about its effects. Commercial settings and scenarios refer to therapies that have been approved by the regulatory agency for one or more indications, e.g., which have been proven to treat the stated indication and have a relatively standardized manufacturing process. For example, in the health care facility system 152 or other external systems, the patient data may be taken from a paper chart or an EHR and manually entered into their patient services system via a “swivel chair” method, e.g., most often a portal. A swivel chair method or interface is a system for computer input and interaction that requires users to move between multiple user interfaces to input and/or process data. While this manner of creating patient data (patient intake) may be inefficient, patient intake is a critical point for clinical and commercial management in personalized medicine because it is the first step to ensuring that the right patient gets the right treatment. For example, in clinical settings, patient data is usually entered into the patient services systems at the patient recruitment stage. If patients pass the eligibility criteria for the screening stage of the trial, they are enrolled and their COI continues. In commercial settings, for example, patients that are entered into the patient services systems are slated to receive treatment; and there may be no requirements upstream of ordering to collect patient data. Differences among each of the various external patient data management platforms, as well as the above differences between such platforms in clinical and commercial settings, require a way to obtain, amalgamate, and harmonize the patient data in a unified format.

The disclosed Patient API 311 is configured to automatically receive patient records from various external platforms and push the patient data in such patient records to the enterprise software platform 100, e.g., allowing HCPs and case managers who manage patients via patient services systems to input patient data into their existing systems external to the enterprise software platform 100, while also preventing the need for duplicate data entry and lowering the probability of human error. This lowers the barrier to entry for ordering treatments with the enterprise software platform 100.

The Patient API 311 can be designed for multiple use cases. For example, the Patient API 311 can batch create and update patients in the implementation process of the enterprise software platform 100. After the implementation phase is complete, the external system can send a list of patients to the enterprise software platform 100 to create, such that upon successful creation, an Ordering user can see the list of patients on the list. The ordering user can then create an order for a patient, and the patient information page will be pre-populated, based on the patient data provided via the Patient API 311.

In some implementations, the Patient API 311 can be configured to manage data transactions between the enterprise software platform 100 and the external system(s) 150 that include a patient identifier (ID), and (optionally) a site identifier associated the ordering institution, and (optionally) patient data including the patient's name, birthdate, biological sex and/or identified gender, and/or contact information (such as email address, phone number, etc.), and/or custom health data (such as weight, height, etc.). These data can be string data for the Patient API 311. The Patient API 311 can include patient or site identifier attributes, which can be objects or an array of objects for the Patient API 311.

In some embodiments of the Patient API 311, the only required field to create a patient is a resource identifier, representing one or more identifiers (e.g., Medical Record Number, Hospital ID) that are associated with the patient. In this manner, the Patient API 311 is able to provide maximum flexibility to patient management users in external systems, for example, as all information about a patient may not be known when they are added to the system. These details can be added later via patient update, for example.

By default, Patient API 311 requires the patient identifiers to be unique. For example, if a user tries to create a patient with the same identifier as an existing patient, the request will fail. In this case, the user should instead update the existing patient with the new details. This prevents the user from creating duplicate patients.

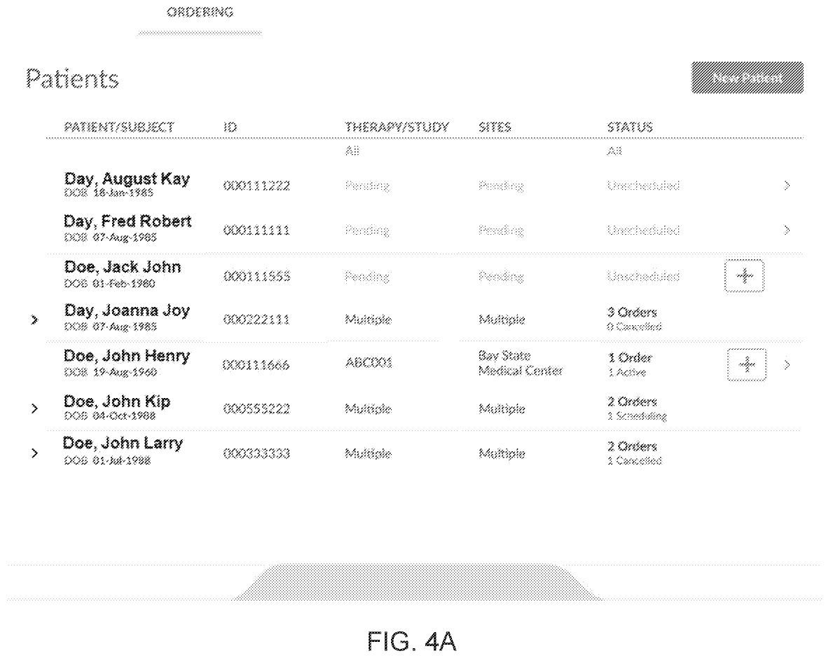

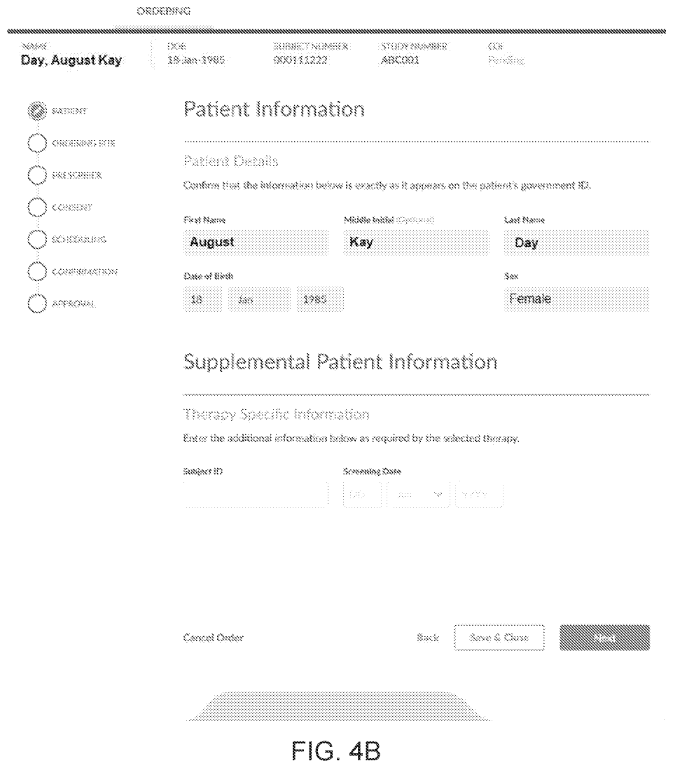

FIGS. 4A and 4B show diagrams of user interfaces associated with the Patient API 311 that are displayable on the client computer device 14 of the external system 150, such as the health care facility system 152. Referring first to FIG. 4A, the example user interface illustrates a list of patients for a therapy order, and the user interface allows selection of listed patients by their name and patient ID, as well as displaying the listed patients' order status, therapy statuses, and site status. The example user interface allows a client user to input a new patient, e.g., by selecting a “New Patient” button. The example user interface provides a graphical data entry and display interface, behind which the Patient API 311 manages the data transfer between the client computer device 14 and the computing devices of the enterprise software platform 100. Referring next to FIG. 4B, the example user interface illustrates another data entry and display interface producible by the Patient API 311 to manage patient data updates (e.g., in a patient information section) and therapy-specific information. Ordering API

Currently, most clinical and commercial therapy developers use their own patient services systems as a single point of access for patient intake, order creation, and order tracking. Clinical and commercial therapies typically have different sets of patient services systems associated with them. In general, these entities have had to rely on multiple patient services systems to manage the supply chain, such as web-based portals for patient recruiting and enrollment, Interactive Web Response Systems (IWRS) and Interactive Voice Response Systems (IVRS) for randomization and assignment, and/or Clinical Trial Management System (CTMS) for treatment journey tracking. These external systems lack a streamlined approach for supply chain management at every node in the supply chain, which, for a personalized therapy, risks disruptions in the ordering and scheduling of the therapy.

The Ordering API 313 of the enterprise software platform 100 provides standard integrations to facilitate a unified experience with little to no duplication of data entry between multiple external systems. Using the APIs 110, the enterprise software platform 100 is configured to initiate and “own” the Chain of Identity process, e.g., by passing all required order-level data to the enterprise software platform 100, via the Ordering API 313, to generate a COI and start the product journey in the platform 100; by providing the user with the ability to create and modify the schedule, via the Scheduling API 315 for orchestrating the schedule of events in the supply chain on the back-end; and by providing the user with the ability to reschedule a shipment leg via a user interface, which can include a user interface produced by the enterprise software platform 100, allowing the user to access and make use of frontend supply chain orchestration features of the platform 100.

The Ordering API 313 facilitates order management integration (OMI) to allow a user to create an approved order in the enterprise software platform 100, where the user is using an external system's user interface (UI). The Ordering API 313 can be configured as an asynchronous pattern API. In some implementations, for example, after creating a patient in the external system 150 (e.g., sent to the enterprise software platform 100 via the Patient API 311), the user can proceed through his/her workflow to supply all the required data to platform 100 in order to create and schedule an order, via the Ordering API 313. In some implementations, for example, the order is sent to the enterprise software platform 100 after this workflow has been completed; put another way, the Ordering API 313 allows the enterprise software platform 100 to receive an order that is already approved and ready to go.

In some implementations, the Ordering API 313 can be configured to manage data transactions between the enterprise software platform 100 and the external system(s) 150 for creating and managing an order, in which the data for the Ordering API 313 includes (i) a patient reference, (ii) resource identifier data, (iii) product data, (iv) ordering institution (site) data, (v) regional data (e.g., US, EU, etc.) associated with the product or treatment, (vi) ordering capabilities data (e.g., functional abilities of a site, such as a collection capability to obtain a sample from a patient), (vii) milestone data, and/or (viii) configurable workflow criteria. In some implementations of the Ordering API 313, the resource identifier data and the milestone data are configured as an array of objects, in which the resource identifier data includes one or more resource identifiers (e.g., strings) associated with an order, and in which the milestone data can include site data, capability data, dates, and other data associated with a milestone of the order. In some implementations, for example, the data for the Ordering API 313 can further include product indication data, e.g., such as a data string identifying one or more diseases or conditions the product is designed (and approved) to treat. In some implementations applicable to clinical scenarios, for example, the data for the Ordering API 313 can further include study data, e.g., such as a data string identifying a study number for a therapy in clinical trials. The data for which the Ordering API 313 manages transactions are associated with fields in the API 313 that are used to look up the corresponding execution context for an order.

In some implementations, for example, a user may have additional attributes that are needed to further filter the list of possible execution contexts for an order. An example of an additional attribute can include a cryopreservation location. Cryopreservation could take place at the apheresis site, a satellite lab, or the manufacturing site, for example. Cryopreservation depends on supply chain optimization factors internal to the customer. To account for this further level of filtering that is needed, a set of configurable workflow criteria, also referred to as workflow variants, can be sent with the request to create an order via the Ordering API 313. Each customer may have a different set of criteria, depending on the selected business process(es). The set of configurable workflow criteria is used as an input to a process that determines which execution context within the initial subset maps to the product journey required for that order.

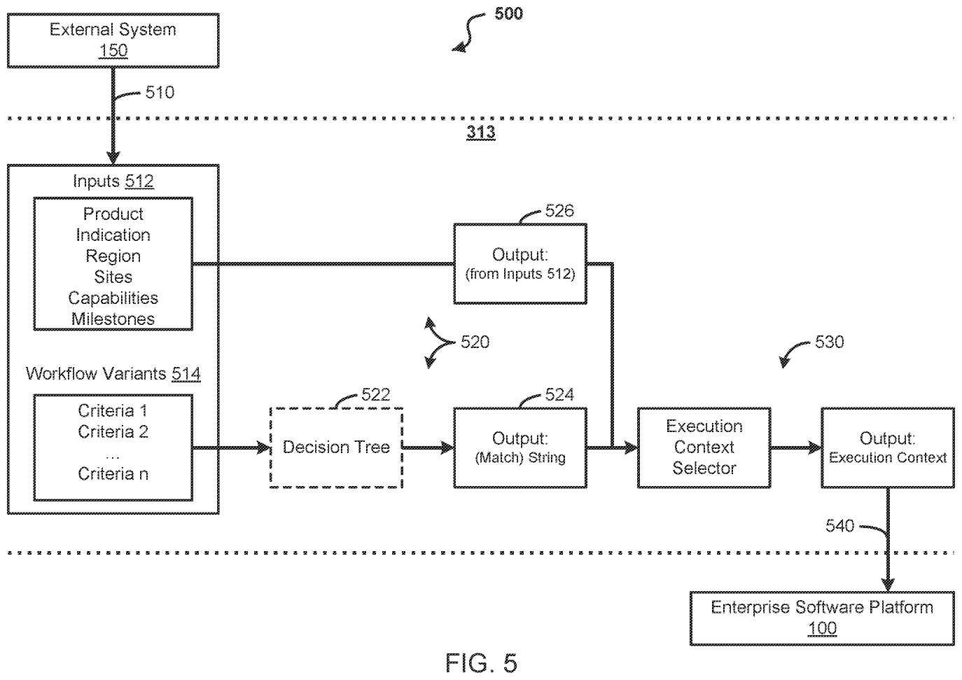

FIG. 5 shows a diagram of an example implementation for an execution method 500 of an order operation by the Ordering API 313. At a process 510 of the method 500, the Ordering API manages inputs 512 and workflow criteria or variants 514, which can be entered by a user, e.g., on a user interface produced by the external system 150 to which the Ordering API is interfaced, or on a user interface associated with the enterprise software platform 100. The inputs 512 can include product data, indication data, region data, ordering institution (site) data, ordering capabilities data, and/or milestones data. The workflow variants 514 can include a plurality of differentiating factors for a product journey of a customer that are not accounted for by product, indication, or region data. In various implementations, the workflow variants 514 can be configured as a data object; and in some implementations, for example, the workflow variants 514 can be configured with an accompanying match string decision tree to be executed in the order.

For the process 510, after entering all ordering and scheduling details in the external system 150, the user will submit the information for the Ordering API 313 to transform and provide the enterprise software platform 100. At a process 520 of the method 500, the inputs 512 are processed by the Ordering API 313 to produce a formatted output of the inputted data from the external system 150. Also, at the process 520, the workflow variants 514 are processed by the Ordering API 313 to produce an output 524 of the data as a string. In some embodiments of the method 500, for example, the workflow variants 514 can be configured within the process 520 using an accompanying match string decision tree 522 to produce the output 524 as a match string. At a process 530 of the method 500, the Ordering API 313 selects the execution context based on the outputted data 526 from the input 512 and outputted data 524 from the workflow variants 514. For example, at the process 530, the Ordering API 313 produces an output data set 538 that is readable and processable by the enterprise software platform 100. At process 540 of the method 500, the Ordering API 313 transfers the output data set 538 to the enterprise software 540.

In some implementations, additional processes in the order operation can be included. For example, some customers may have additional ordering steps between a personalized therapy ordering nurse and the supply chain management (enterprise software platform 100), such as reimbursement activities. The Ordering API 313 is designed to provide flexibility to incorporate client-desired steps in the ordering, such as for this use case or other use cases.

In some embodiments, for example, the Ordering API 313 is configured as an asynchronous API, e.g., where the system will simply receive a GraphQL response with a transaction UUID. The Ordering API 313 can be set up to be listening to event notifications (e.g., via webhook), to which the enterprise software platform 100 can send success or failure event notifications with the associated transaction UUID for correlation.

In example implementations where the method 500 is for executing an order creation, at the process 540, a request to create an order that is sent to the enterprise software platform 100 can cause the enterprise software platform 100 to create the required data models, select the product journey, schedule the order, approve the order, and kick off the collection procedure. In some implementations, this order can be automatically approved, and downstream operations are automatically executed by the enterprise software platform 100.

In example implementations where the method 500 is for executing an order cancelation, at the process 510, a user is also able to cancel an order from their external system 150 via Ordering API 313. As an example use case, an order for a prescribed personalized therapy will need to be canceled in the event that a patient has an adverse event or no longer desires treatment. As another example use case, a cancelation can occur when an order needs to be rescheduled to follow a different product journey than was originally scheduled. In some implementations, the method 500 can be implemented for updating order fields to support an updated execution context for a modified/rescheduled order; whereas in other implementations, for example, the method 500 can be implemented for canceling the present order and creating a new order with the rescheduling information. In this case, an order cancelation event notification can be generated by the enterprise software platform 100 and delivered via the Ordering API 313 to the external system 150 and/or delivered by another communication manner to the external system 150 so that the system 150 is aware of the cancelation.

### Scheduling API

In some implementations, the Scheduling API 315 can be configured to manage data transactions between the enterprise software platform 100 and the external system(s) 150 that include an order reference (e.g., which includes the order's associated identifier), the patient ID, a treatment reference (e.g., which includes the order's associated identifier), and/or milestone data, including a name associated with the milestone, a data/time associated with when the milestone is to occur, and/or a milestone site or location.

The Schedule API 315 creates or updates the schedule of a treatment order when a user of the client computer device 14 of the external system 150 executes a schedule call, e.g., inputs a schedule creation or update command or data entry via a user interface designed to interact with the Schedule API 315. The Schedule API 315 will check if the treatment already exits; and if so, that treatment will be updated with the parameters provided in the schedule call, and if not, a schedule for that treatment will be created with the parameters provided in the schedule call. A schedule call allows users to update a milestone or multiple milestones to a treatment as necessary.

As an illustrative example, a patient is created via the Patient API 311 and an order is then created via the Ordering API 313, in which a treatment is scheduled via the Scheduling API 315. A week later, the patient needs to reschedule their collection time. The HCP user, at the client computer device 14 of the health care facility system 152, can generate a schedule call to pass only the changed milestone, e.g., such as a different date for the treatment, and the Scheduling API 315 would manage data transaction with a scheduler module of the enterprise software platform 100 to recalculate the treatment schedule accordingly. For example, the response for the Scheduling API 315 can return information about all milestones, even if only one milestone was defined or changed in the request.

In some implementations, for example, the Scheduling API 315 can create a pending schedule for a treatment, which will not impact the timeline of events for stakeholders unless it is confirmed. For example, if a user (e.g., nurse) of the client computer device 14 of the health care facility system 152 created an order and selected the most convenient day for the patient to undergo apheresis, but based on an internal process of the health care facility system 152 the date needs to be reviewed by case management, an endpoint can inform the Scheduling API 315 to manage the creation of a pending schedule that can be confirmed or rejected. In such cases, unless the pending schedule is confirmed, changes would not impact the treatment timeline. For example, if the case manager of the health care facility system 152 saw the pending schedule request from the apheresis nurse and, during review, found that the update request would not introduce issues connected to patient safety, the case manager could confirm the pending schedule and all the changes will be applied, where the Scheduling API 315 will facilitate creation of a new timeline accordingly. On the other hand, for example, if the case manager saw the pending schedule request from the apheresis nurse and, upon review, found some limitations or risk to the patient's safety, the case manager could reject the pending schedule via input to the Scheduling API 315, which would not confirm the pending timeline, and hence not impact the original treatment timeline.

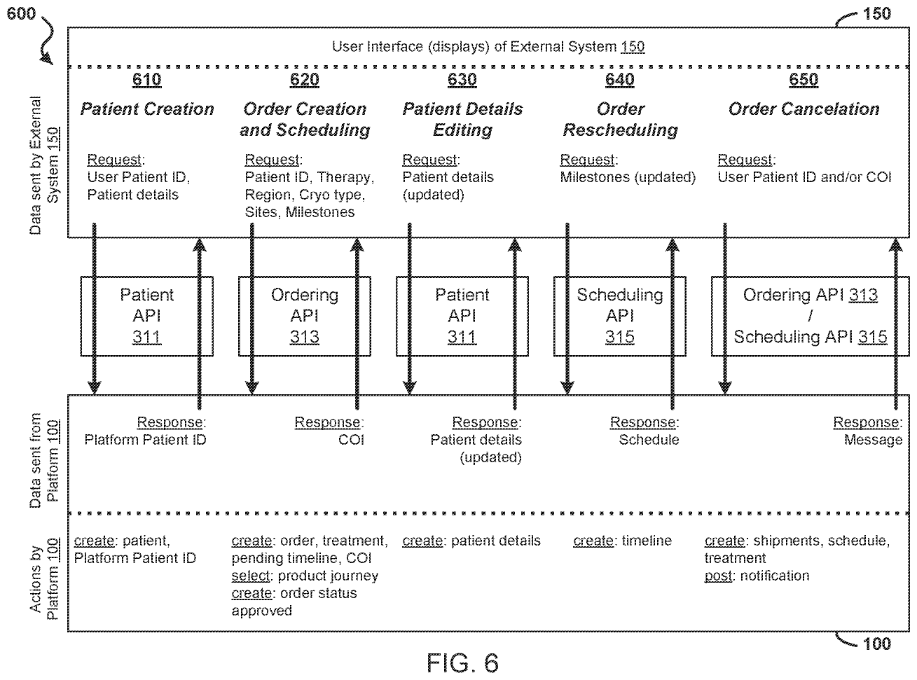

FIG. 6 shows a diagram of illustrating data transactions 600 managed by the set of APIs 110 of the enterprise software platform 100 based on data provided by the external system 150. In the example implementation shown in FIG. 6, a user using a user interface of the external system 150 inputs data for an action 610 to create a patient 610, for an action 620 to create a (personalized therapy treatment) order and schedule the (personalized therapy treatment) events, for an action 630 to edit patient details, for an action 540 to reschedule (one or more events or aspects of the scheduled personalized therapy treatment), and for an action 650 to cancel the order. In some implementations, for example, some of the data transactions 600 are implemented using the set of APIs 110, as certain data transactions such as patient editing, order rescheduling, and/or order cancelation are not necessary for a particular individualized patient treatment.

In the action 610, the external system 150 creates a request to create a patient that includes a patient ID associated with the external system 150 (and optionally patient details). The Patient API 311 receives and processes the request with the input data and produces an output processible by the enterprise software platform 100 to produce one or more actions. In response to the request (provided by the Patient API 311) for the action 610, the enterprise software platform 100 can create a patient in the data system (e.g., database 16) of the platform 100, including creating a platform patient ID for the platform 100 that is mapped to the patient ID associated with the external system 150. The Patient API 311 can transmit a response to the external system 150, e.g., providing data including the platform patient ID.

In the action 620, the external system 150 creates a request to create a personalized therapy treatment order and schedule the personalized therapy treatment events, in which the request includes the patient ID associated with the external system 150, therapy data, order site data, region data, and milestone data (and optionally custom data (criteria), such as a cryogenic storage, for example). The Ordering API 313 receives and processes the request with the input data (and workflow variant information) and produces an output processible by the enterprise software platform 100 to produce one or more actions. In response to the request (provided by the Ordering API 313) for the action 620, the enterprise software platform 100 can create an order for the treatment and a chain of identity (COI), which includes order data, treatment data, COI for the patient, and a pending timeline, in the data system (e.g., database 16) of the platform 100. An example of the created COI identifier is shown in Table 2. Also, the enterprise software platform 100 can select the product journey and create an order status identifier as approved. The Ordering API 313 can transmit a response to the external system 150, e.g., providing data including the COI.

In the action 630, the external system 150 creates a request to edit patient details, e.g., which could include adding or amending patient data such as a patient's name, birthday, contact information, etc. The Patient API 311 receives and processes the request with the input data and produces an output processible by the enterprise software platform 100 to produce one or more actions. In response to the request (provided by the Patient API 311) for the action 630, the enterprise software platform 100 can input the data in the created patient associated with the platform patient ID in the data system (e.g., database 16) of the platform 100. For example, in some implementations, as part of the data transactions implemented by the Patient API 311 after receiving the request from the external system 150, the Patient API 311 can query the enterprise software platform 100 with the patient ID to obtain the platform patient ID from the patient record stored on the data system of the platform 100, which the platform patient ID can be used by the Patient API 311 to interface with various services internal to the platform 100 to update the patient data. As an example, the platform 100 can format the patient data to conform with constraints and/or requirements associated with that particular patient entry based on the platform patient ID. The Patient API 311 can transmit a response to the external system 150, e.g., providing data including the updated patient details.

In the action 640, the external system 150 creates a request to reschedule the personalized therapy treatment, in which the request includes milestone data (updated). The Scheduling API 315 receives and processes the request with the updated milestone data and produces an output processible by the enterprise software platform 100 to produce one or more actions. In response to the request (provided by the Scheduling API 315) for the action 640, the enterprise software platform 100 can create change a timeline of the events in the personalized therapy treatment schedule in the data system (e.g., database 16) of the platform 100, e.g., creating a modified timeline. The Scheduling API 315 can transmit a response to the external system 150, e.g., providing data including the updated schedule data.

In the action 650, the external system 150 creates a request to cancel the order for the personalized therapy treatment, in which the request includes the patient ID and/or the COI. The Ordering API 313 and the Scheduling API 315 receive and process the request with the patient ID and/or COI and produce respective outputs processible by the enterprise software platform 100 to produce one or more actions. In response to the request (provided by the Ordering API 313 and/or the Scheduling API 315) for the action 650, the enterprise software platform 100 can create shipments, schedule and treatment cancelations in the data system (e.g., database 16) of the platform 100. In some implementations of the action 650, for example, the Ordering API 313 queries the order that has been requested to be cancelled and verifies it is in a state where it can be cancelled; the Ordering API 313 then sends requests to the workflow engine to cancel all in-flight processes (e.g., sample collection, manufacturing, and/or delivery-treatment infusion). Further, the Ordering API 313 can append a “cancelled” flag on backend models and can send a request to the Scheduling API 315 to cancel the order's schedule and shipments. In such implementations, the Scheduling API 315 requests the internal shipment service to cancel all shipments for the order. The Ordering API 313 can transmit a response to the external system 150, e.g., providing a notification that the order was canceled.

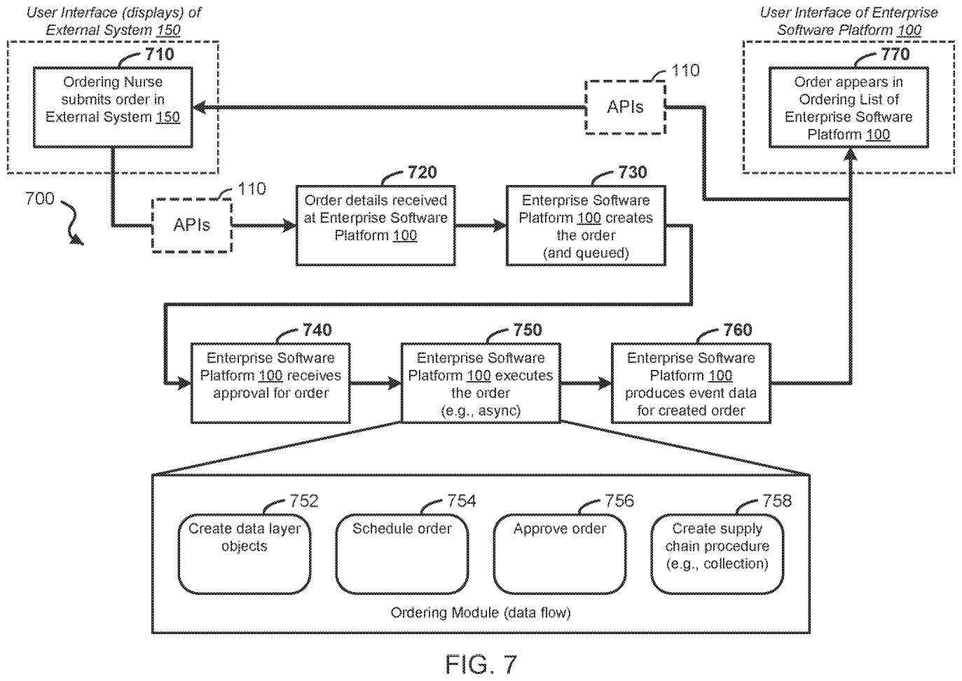

FIG. 7 shows a diagram of an example embodiment of a method 700 for creating a treatment workflow by the enterprise software platform 100 based on an order request submitted by the external system 150. The method 700 includes a process 710 for a user (e.g., ordering nurse using a client computing device 14 of the health care facility system 152) to submit an order request for a personalized therapy treatment for a patient. The method 700 includes a process 720 to process the order request by the APIs 110 (e.g., Ordering API 313) to provide order details to the enterprise software platform 100 (e.g., data from the order request structured for the enterprise software platform 100). The method 700 includes a process 730 to create the order at the enterprise software platform 100 based on the received order details. The method 700 includes a process 730 to receive approval data and process an approval of the order, e.g., by the APIs 110, to provide order approval to the enterprise software platform 100. In some implementations, for example, the order can be created by the platform 100 at the process 730 without the order having been approved, in which the order is created but the method 700 is not able to continue processing the unapproved order until approval has been received; in such implementations, for example, the created but unapproved order may sit in a queue and await approval. Upon receiving and processing approval at the process 740, the method 700 includes a process 750 to execute the order at the enterprise software platform. In some embodiments of the process 750, the process 750 includes: a process 752 to create data layer objects for data associated with the personalized therapy treatment; a process 754 to create a scheduling of the personalized therapy treatment; a process 756 to process approval of the order; and a process 758 to create one or more supply chain procedures for the personalized therapy treatment, e.g., such as a collection procedure, a manufacturing procedure a delivery procedure, etc. The method 700 includes a process 760 to produce event data for the created order at the enterprise software platform 100. The method 700 includes a process 770 to provide created-order data displayable on a user interface. In implementations of the process 770, a display device (e.g., client computer device 14 of the enterprise software platform 100 and/or client computer device 14 of the external system 150) receives the created-order data and renders a user interface to be displayed. In some implementations of the process 770, the client computer device 14 of the external system 150 can receive displayable data corresponding to the created-order data by the APIs 110 processing the created-order data to structure the created-order data that can be rendered by the external system 150 for display on a user interface.

### Example Computing Devices

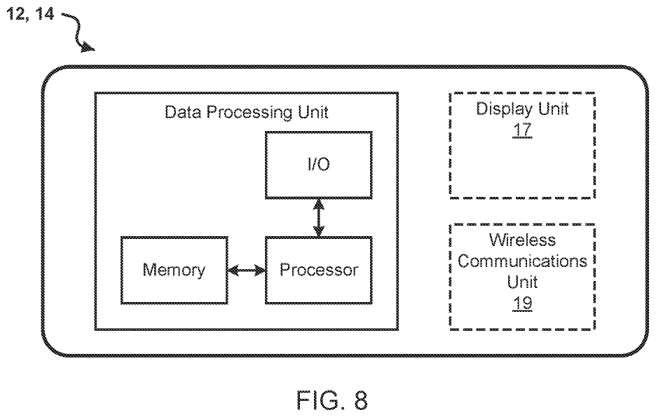

FIG. 8 shows a block diagram of an example embodiment of the computer device 12 and 14 in the environment 10 of FIG. 1. In various implementations, the computing devices 12, 14 can include a server computing device, a desktop or laptop computing device, a mobile computing device such as a smartphone, tablet, smartwatch or other wearable device, and other suitable type of computing device. As shown in FIG. 8, the computer device 12, 14 includes a data processing unit and, optionally, a display unit and/or a wireless communication unit. For example, the computer device 12, 14 can be implemented as a client computer. Also, for example, the computer device 12, 14 can be implemented as a server computer. The data processing unit of the computer device 12, 14 includes one or more processors operable to process data, one or more memories in communication with the processor to store data, and an input/output unit (I/O) to interface the processor and/or memory to other software or hardware modules, units, or devices of the computer device 12, 14 or an external device. For example, the one or more processors can include a central processing unit (CPU), a graphical processing unit (GPU), a microcontroller unit (MCU) or other processor. For example, the one more memories can include and store processor-executable code, which when executed by the processor(s), configures the data processing unit to perform various operations, e.g., such as receiving information, commands, and/or data, processing information and data, and transmitting or providing information/data to another device. In some implementations, the data processing unit can transmit raw or processed data to a computer system or communication network accessible via the Internet (referred to as ‘the cloud’) that includes one or more remote computational processing devices (e.g., servers in the cloud). To support various functions of the data processing unit, the one or more memories can store information and data, such as instructions, software, values, images, and other data processed or referenced by the processor(s). For example, various types of Random Access Memory (RAM) devices, Read Only Memory (ROM) devices, Flash Memory devices, and other suitable storage media can be used to implement storage functions of the one or more memories. In some embodiments of the computer device 12 and 14, for example, the computer device 12, 14 can optionally include a wireless communication unit 19, which can be in data communication with the data processing unit via the I/O. The I/O of the data processing unit can interface the data processing unit to utilize various types of wired or wireless interfaces compatible with typical data communication standards, for example, which can be used in communications of the data processing unit with other devices, via a wireless transmitter/receiver (Tx/Rx) unit, e.g., including, but not limited to, Bluetooth, Bluetooth low energy (BLE), Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE/5G cellular communication methods, NFC (Near Field Communication), and parallel interfaces. The I/O of the data processing unit can also interface with other external interfaces, sources of data storage, and/or visual or audio display devices, etc. to retrieve and transfer data and information that can be processed by the processor(s), stored in the memory unit, or exhibited on an output unit of the computer device 12, 14 or an external device. In some embodiments of the computer device 12 and 14, for example, the computer device 12, 14 can optionally include a display unit 17 that is in data communication with the data processing unit, e.g., via the I/O, to provide a visual display, an audio display, and/or other sensory display that produces the user interface of the software application in accordance the disclosed technology. In some examples, the display unit 17 can include various types of screen displays, speakers, or printing interfaces, e.g., including but not limited to, light emitting diode (LED), or liquid crystal display (LCD) monitor or screen, cathode ray tube (CRT) as a visual display; audio signal transducer apparatuses as an audio display; and/or toner, liquid inkjet, solid ink, dye sublimation, inkless (e.g., such as thermal or UV) printing apparatuses, etc.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

### Examples

Several aspects of the present technology are set forth in the following examples. Although several aspects of the present technology are set forth in examples directed to systems, computer-readable mediums, and methods, any of these aspects of the present technology can similarly be set forth in examples directed to any of systems, computer-readable mediums, and methods in other embodiments.
1. An enterprise software platform for orchestrating a personalized therapy development and/or treatment order in a supply chain, the enterprise software platform comprising:
   one or more processors; and
   one or more storage devices coupled to the one or more processors and configured to store instructions that, when executed by the one or more processors, cause the enterprise software platform to perform operations comprising:
      transmitting data to and receiving data from one or more remote computing devices operating a software system external to the enterprise software platform,
      wherein the data transmitted to and received from the one or more remote computing devices is passed through a set of application program interfaces (APIs), including a patient API that manages data transactions associated with patient data, an ordering API that manages data transactions associated with personalized therapy treatment order data, and a scheduling API to manage data transactions associated with personalized therapy treatment schedule data.
2. The enterprise software platform of example 1 wherein the patient API is configured to structure the patient data from a first data structure associated with the external software system to a second data structure readable and processible by the enterprise software platform.
3. The enterprise software platform of example 1 or 2 wherein the patient API is configured to facilitate creation of a new patient data record in the enterprise software platform that includes a platform patient identification (ID) that corresponds to an external system patient ID included in the patient data.
4. The enterprise software platform of any of examples 1-3 wherein the patient data received from the external system includes the external system patient ID, ordering institution information associated with the external software system, and patient information.
5. The enterprise software platform of example 4 wherein the enterprise software platform is configured to restrict access to the patient information to a user that does not possess ordering institution information of the external software system.
6. The enterprise software platform of any of examples 1-5 wherein the ordering API is configured to structure the personalized therapy treatment order data from a third data structure associated with the external software system to a fourth data structure readable and processible by the enterprise software platform.
7. The enterprise software platform of example 6 wherein the ordering API is configured to create, modify, and/or cancel the personalized therapy development and/or treatment order in the supply chain in ordering data in the enterprise software platform, wherein the ordering data includes one or more of (i) a patient reference, (ii) product information, (iii) ordering institution information associated with the external software system, (iv) regional information associated with location of the product or treatment, (v) indication data, (vi) milestone data, and (vii) configurable workflow criteria that are configurable by a user of the external software system.
8. The enterprise software platform of example 7 wherein the ordering API is configured to automate selection of a workflow of actions in the supply chain among a plurality of workflows, which are stored by the enterprise software platform, by utilizing the configurable workflow criteria to include attributes of the personalized therapy development or treatment order to select a most appropriate workflow from the plurality of workflows.
9. The enterprise software platform of any of examples 1-8 wherein the scheduling API is configured to structure the schedule data from a fifth data structure associated with the external software system to a sixth data structure readable and processible by the enterprise software platform.
10. The enterprise software platform of example 9 wherein the scheduling API is configured to create, update, or cancel a schedule of milestones for the personalized therapy development and/or treatment order in the supply chain.
11. The enterprise software platform of example 10 wherein the schedule data includes an order reference, external system patient ID, a treatment reference, and/or milestone data.
12. A method for orchestrating a personalized therapy development and/or treatment order in a supply chain, the method comprising:
   from a server, transmitting data to and receiving data from one or more remote computing devices operating a software system external to the enterprise software platform,
   wherein the data transmitted to and received from the one or more remote computing devices is passed through a set of application program interfaces (APIs), including—
      a patient API that manages data transactions associated with patient data,
      an ordering API that manages data transactions associated with personalized therapy treatment order data, and
      a scheduling API to manage data transactions associated with personalized therapy treatment schedule data.
13. The method of example 12 wherein the patient API is configured to facilitate creation of a new patient data record in the enterprise software platform that includes a platform patient identification (ID) that corresponds to an external system patient ID included in the patient data.
14. The method of example 12 or example 13 wherein the patient data received from the external system includes the external system patient ID, ordering institution information associated with the external software system, and patient information.
15. The method of example 14 wherein the enterprise software platform is configured to restrict access to the patient information to a user that does not possess ordering institution information of the external software system.
16. The method of any of examples 12-15 wherein the ordering API is configured to structure the personalized therapy treatment order data from a third data structure associated with the external software system to a fourth data structure readable and processible by the enterprise software platform.
17. The method of any of examples 12-16 wherein:
   the ordering API is configured to create, modify, and/or cancel the personalized therapy development and/or treatment order in the supply chain in ordering data in the enterprise software platform; and
   the ordering data includes one or more of (i) a patient reference, (ii) product information, (iii) ordering institution information associated with the external software system, (iv) regional information associated with location of the product or treatment, (v) indication data, (vi) milestone data, and (vii) configurable workflow criteria that are configurable by a user of the external software system.
18. The method of any of examples 12-17 wherein the ordering API is configured to automate selection of a workflow of actions in the supply chain among a plurality of workflows, which are stored by the enterprise software platform, by utilizing the configurable workflow criteria to include attributes of the personalized therapy development or treatment order to select a most appropriate workflow from the plurality of workflows.
19. The method of any of examples 12-18 wherein:
   the schedule data includes an order reference, external system patient ID, a treatment reference, and/or milestone data; and
   the scheduling API is configured to create, update, and/or cancel a schedule of milestones for the personalized therapy development and/or treatment order in the supply chain.
20. A non-transitory, computer-readable medium storing instructions thereon that, when executed by one or more processors of a computing system, cause the computing system to perform operations for orchestrating a personalized therapy development and/or treatment order in a supply chain, the operations comprising:
   transmitting data to and receiving data from one or more remote computing devices operating a software system external to the enterprise software platform,
   wherein the data transmitted to and received from the one or more remote computing devices is passed through a set of application program interfaces (APIs), including a patient API that manages data transactions associated with patient data, an ordering API that manages data transactions associated with personalized therapy treatment order data, and a scheduling API to manage data transactions associated with personalized therapy treatment schedule data.

### CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. As another example, various components of the technology can be further divided into subcomponents, and/or various components and/or functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology.

It should also be noted that other embodiments in addition to those disclosed herein are within the scope of the present technology. For example, embodiments of the present technology can have different configurations, components, and/or procedures in addition to those shown or described herein. Moreover, a person of ordinary skill in the art will understand that these and other embodiments can be without several of the configurations, components, and/or procedures shown or described herein without deviating from the present technology. Accordingly, the disclosure and associated technology can encompass other embodiments not expressly shown or described herein.

I/We claim:

1. An enterprise software platform for orchestrating a personalized therapy development and/or treatment order in a supply chain, the enterprise software platform comprising:
one or more processors; and
one or more storage devices coupled to the one or more processors and configured to store instructions that, when executed by the one or more processors, cause the enterprise software platform to perform operations comprising:
transmitting data to and receiving data from one or more remote computing devices operating a software system external to the enterprise software platform,
wherein the data transmitted to and received from the one or more remote computing devices is passed through a set of application program interfaces (APIs), including a patient API that manages data transactions associated with patient data, an ordering API that manages data transactions associated with personalized therapy treatment order data, and a scheduling API to manage data transactions associated with personalized therapy treatment schedule data.

2. The enterprise software platform of claim 1 wherein the patient API is configured to structure the patient data from a first data structure associated with the external software system to a second data structure readable and processible by the enterprise software platform.

3. The enterprise software platform of claim 2 wherein the patient API is configured to facilitate creation of a new patient data record in the enterprise software platform that includes a platform patient identification (ID) that corresponds to an external system patient ID included in the patient data.

4. The enterprise software platform of claim 1 wherein the patient data received from the external system includes the external system patient ID, ordering institution information associated with the external software system, and patient information.

5. The enterprise software platform of claim 4 wherein the enterprise software platform is configured to restrict access to the patient information to a user that does not possess ordering institution information of the external software system.

6. The enterprise software platform of claim 1 wherein the ordering API is configured to structure the personalized therapy treatment order data from a third data structure associated with the external software system to a fourth data structure readable and processible by the enterprise software platform.

7. The enterprise software platform of claim 6 wherein the ordering API is configured to create, modify, and/or cancel the personalized therapy development and/or treatment order in the supply chain in ordering data in the enterprise software platform, wherein the ordering data includes one or more of (i) a patient reference, (ii) product information, (iii) ordering institution information associated with the external software system, (iv) regional information associated with location of the product or treatment, (v) indication data, (vi) milestone data, and (vii) configurable workflow criteria that are configurable by a user of the external software system.

8. The enterprise software platform of claim 7 wherein the ordering API is configured to automate selection of a workflow of actions in the supply chain among a plurality of workflows, which are stored by the enterprise software platform, by utilizing the configurable workflow criteria to include attributes of the personalized therapy development or treatment order to select a most appropriate workflow from the plurality of workflows.

9. The enterprise software platform of claim 1 wherein the scheduling API is configured to structure the schedule data from a fifth data structure associated with the external software system to a sixth data structure readable and processible by the enterprise software platform.

10. The enterprise software platform of claim 9 wherein the scheduling API is configured to create, update, or cancel a schedule of milestones for the personalized therapy development and/or treatment order in the supply chain.

11. The enterprise software platform of claim 10 wherein the schedule data includes an order reference, external system patient ID, a treatment reference, and/or milestone data.

12. A method for orchestrating a personalized therapy development and/or treatment order in a supply chain, the method comprising:
from a server, transmitting data to and receiving data from one or more remote computing devices operating a software system external to the enterprise software platform,
wherein the data transmitted to and received from the one or more remote computing devices is passed through a set of application program interfaces (APIs), including—
a patient API that manages data transactions associated with patient data,
an ordering API that manages data transactions associated with personalized therapy treatment order data, and
a scheduling API to manage data transactions associated with personalized therapy treatment schedule data.

13. The method of claim 12 wherein the patient API is configured to facilitate creation of a new patient data record in the enterprise software platform that includes a platform patient identification (ID) that corresponds to an external system patient ID included in the patient data.

14. The method of claim 12 wherein the patient data received from the external system includes the external system patient ID, ordering institution information associated with the external software system, and patient information.

15. The method of claim 14 wherein the enterprise software platform is configured to restrict access to the patient information to a user that does not possess ordering institution information of the external software system.

16. The method of claim 12 wherein the ordering API is configured to structure the personalized therapy treatment order data from a third data structure associated with the external software system to a fourth data structure readable and processible by the enterprise software platform.

17. The method of claim 12 wherein:
the ordering API is configured to create, modify, and/or cancel the personalized therapy development and/or treatment order in the supply chain in ordering data in the enterprise software platform; and
the ordering data includes one or more of (i) a patient reference, (ii) product information, (iii) ordering institution information associated with the external software system, (iv) regional information associated with location of the product or treatment, (v) indication data, (vi) milestone data, and (vii) configurable workflow criteria that are configurable by a user of the external software system.

18. The method of claim 12 wherein the ordering API is configured to automate selection of a workflow of actions in the supply chain among a plurality of workflows, which are stored by the enterprise software platform, by utilizing the configurable workflow criteria to include attributes of the personalized therapy development or treatment order to select a most appropriate workflow from the plurality of workflows.

19. The method of claim 12 wherein:
the schedule data includes an order reference, external system patient ID, a treatment reference, and/or milestone data; and
the scheduling API is configured to create, update, and/or cancel a schedule of milestones for the personalized therapy development and/or treatment order in the supply chain.

20. A non-transitory, computer-readable medium storing instructions thereon that, when executed by one or more processors of a computing system, cause the computing system to perform operations for orchestrating a personalized therapy development and/or treatment order in a supply chain, the operations comprising:
transmitting data to and receiving data from one or more remote computing devices operating a software system external to the enterprise software platform,
wherein the data transmitted to and received from the one or more remote computing devices is passed through a set of application program interfaces (APIs), including a patient API that manages data transactions associated with patient data, an ordering API that manages data transactions associated with personalized therapy treatment order data, and a scheduling API to manage data transactions associated with personalized therapy treatment schedule data.
